(19) United States
(12) Patent Application Publication
(10) Pub. No.: US 2009/0253650 A1
(43) Pub. Date: Oct. 8, 2009

(54) Teat antiseptic prepared from polysaccharide gel with bactericidal and immuno-stimulating activity isolated from durian fruit-rind

(76) Inventors: S. Pongsamart, Patumwan (TH); Piyarat Chansiripornchai, Patumwan (TH); Kittisak Ajariyakajorn, Patumwan (TH); Raktham Maktrirat, Patumwan (TH)

Correspondence Address: Dr. S. Pongsamart;Faculty of Pharmaceutical Sciences, Chulalongkorn University, Phyathai Road, Patumwan, Bangkok, 10330, TH

(21) Appl. No.: 12/080,280

(22) Filed: Apr. 3, 2008

(51) Int. Cl. A61K 31/715; C07H 3/00; A61P 31/04

(52) U.S. Cl. 514/54; 536/123.1

(57) ABSTRACT

A teat antiseptic having bactericidal activity against bacteria causing bovine mastitis. A post-milking teat dip and teat seal were prepared using polysaccharide gel with bactericidal, immuno-stimulating and wound healing activity isolated from durian fruit rind as active ingredient.

### BACKGROUND OF THE INVENTION

This invention relates to teat antiseptic preparation used as a post-milking teat dip after milking and teat seal used in dry period of dairy cow prepared from polysaccharide gel isolated from durian rind. The preparation is used to prevent bovine mastitis.

Recent research on applications of natural polysaccharide gel has received great attention because these compounds are of high hydrolytic stability, yet are easily biodegradable. The natural polysaccharides isolated from dried fruit-rind of durian are high-molecular weight polysaccharides present in fruit-rind of durian. In addition to antibacterial activity against pus-forming or acne-causing bacteria, it has been shown to exhibit good inhibition against various bacteria causing bovine mastitis. Immuno-stimulating activity of durian polysaccharide gel, at concentration of 0.5-1% w/v, on cell of immune system in mice and especially on bovine mammary leukocytes (BMLs) in dairy cows has been demonstrated. The polysaccharide gel can be used as teat antiseptic in dairy cows to cover nicely the teat with soothing effect having antibacterial activity against bacteria causing bovine mastitis. A post-milking teat dip can be used not only killing bacteria but also healing skin lesion or abrasion at teat-end of milking cows.

### SUMMARY OF THE INVENTION

A water-soluble polysaccharide gel (PG) was extracted from fruit-rind of durian (Durio zibethinus Murr.) comprises steps of boiling dried fruit rind in 25-30 volume of water for 40 minutes, filtering, repeating boiling residue 1-2 times with 15 volume by weight of water. Filtrates were pooled and concentrated by rotary evaporating at 70° C. under reduced pressure and precipitating using acidified aqueous ethanol, collecting precipitate, drying and pulverizing. The polysaccharide gel has very good gelling property including good biological properties such as antibacterial activity against various pathogenic microorganisms causing bovine mastitis and immuno-stimulating activity of bovine mammary leukocytes. Post-milking teat dip preparations are prepared using durian polysaccharide gel. This teat dip preparation has been shown to be effective in protection against bovine mastitis pathogens and also healing teat-end of cows. Teat seal preparations are prepared using durian polysaccharide gel for preventing mastitis infection during dry period in dairy cows.

### BRIEF DESCRIPTION OF THE DRAWINGS

FIG. 1 shows scanning Electron Micrograph of Streptococcus agalactiae after incubation with PG at 37° C. for 8 hours. (A) Non-viable S. agalactiae in NSS with 2.5% PG; (B) S. agalactiae in NSS without PG.

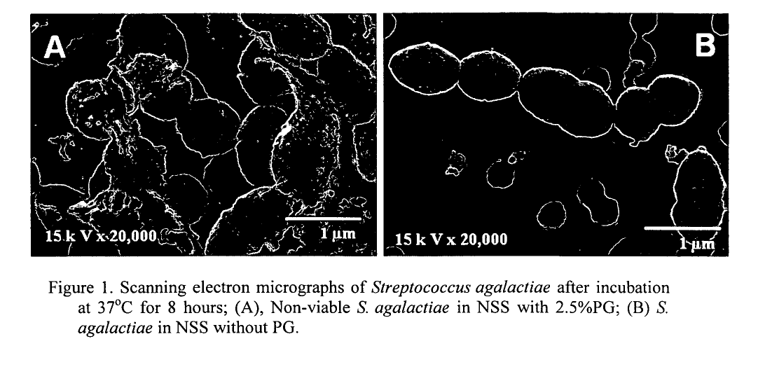

FIG. 2 shows bactericidal activity of PG teat dip against Streptococci causing bovine mastitis; (A) contagious pathogens and, (B) environmental pathogens.

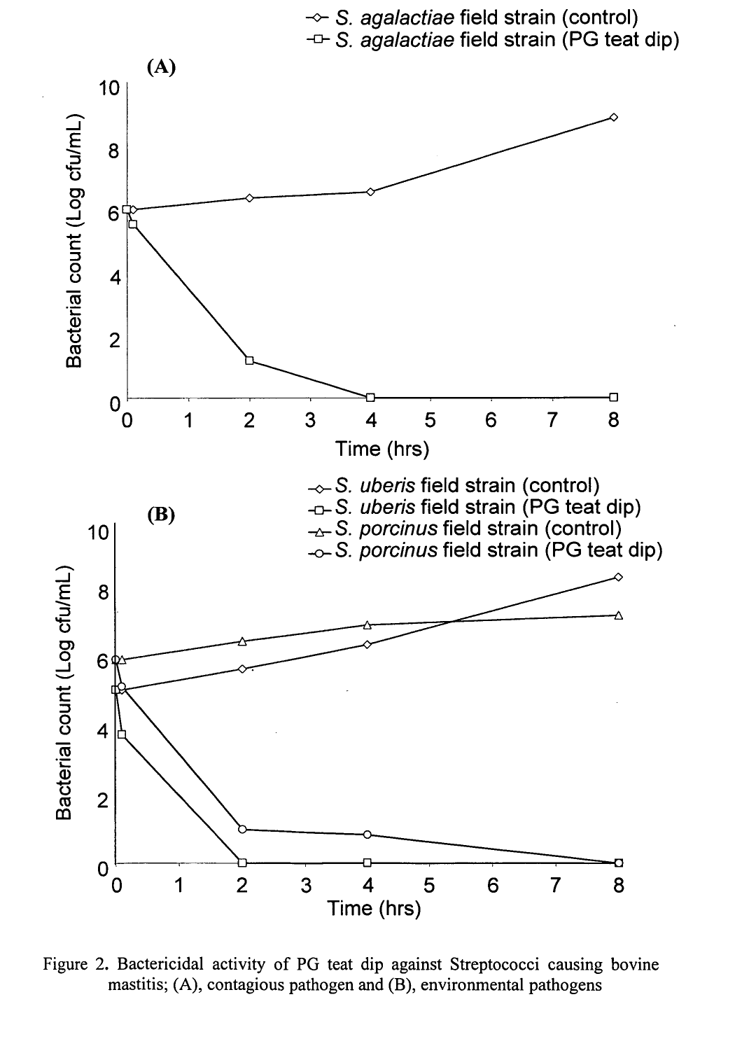

FIG. 3 shows bactericidal activity of PG teat dip against Staphylococci causing bovine mastitis; (A) contagious pathogens and, (B) environmental pathogens.

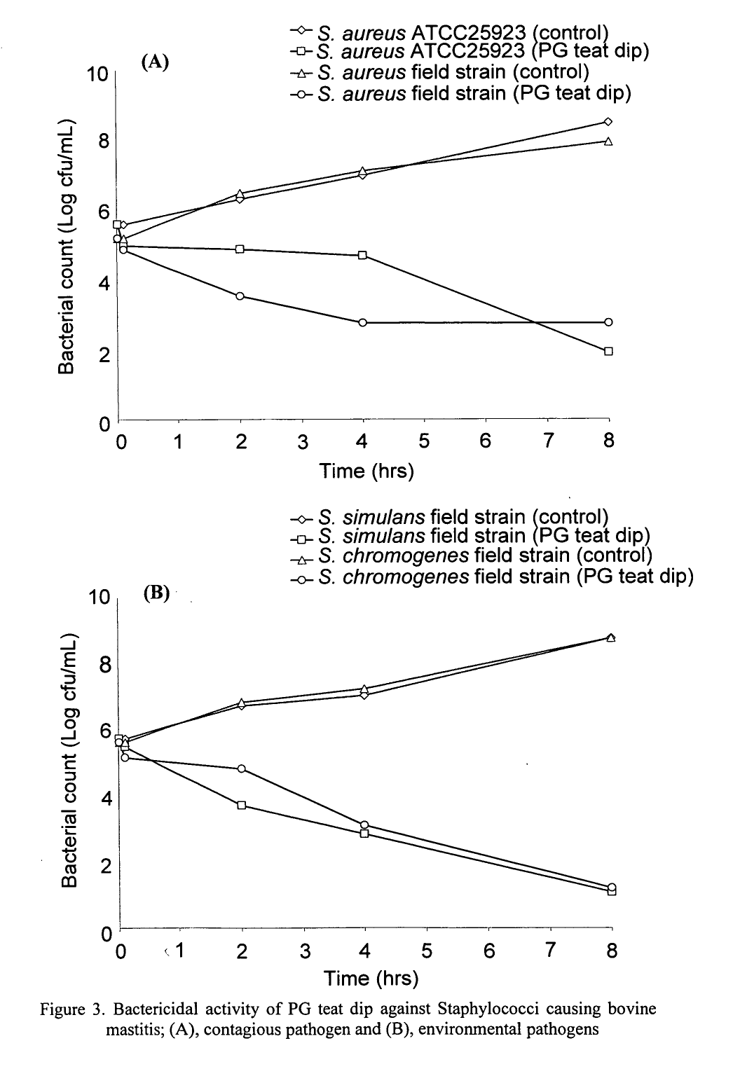

FIG. 4 shows bactericidal activity of PG teat dip against gram-negative bacteria causing bovine mastitis; (A) E. coli; (B) Klebsiella sp. and Pseudomonas sp.

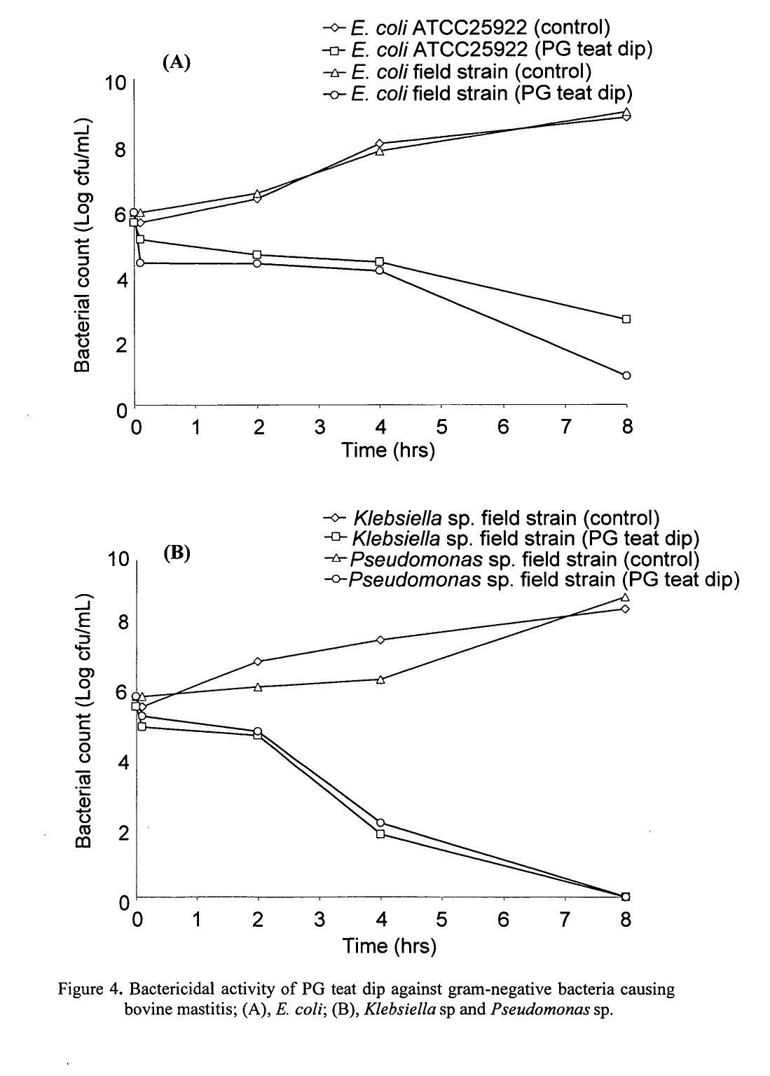

### DETAILED DESCRIPTION OF THE PREFERRED EMBODIMENTS

Polysaccharide gel (PG) was extracted from dried fruit-rind of durian (Durio zibethinus Murr.) a plant cultivated in Thailand with delicious fruit (Pongsamart and Panmaung (1998) Songkhlanakarin J Sci Technol, 20 (3), 323-332). The polysaccharide is characterized to be pectic polysaccharide with immunomodulating activity by complement fixation test (Hokputsa et al. (2004) Carbohydrate Polymers 56:471-481). The gel itself was found to possess good antibacterial activity against various microorganisms i.e. Staphylococcus aureus, Staphylococcus epidermidis, Micrococcus luteus, Bacillus subtilis, Escherichia coli, and Lactobacillus pentosus (Pongsamart et al (2005), Acta Horticulturae 678, 65-73), acne-causing bacterium Propionebacterium acnes ( Paphattarapong et al. (2006) Acta Pharmacol Sin Suppl. 1,1-489,57) , and at least 106 field isolates of mastitis-causing bacteria in dairy cows, especially those contagious pathogens i.e. Staphylococcus aureus and Streptococcus agalactiae, (Lipipun et al (2006) Acta Pharmacol Sin Suppl. 1,1 -489,54). This polysaccharide gel is water-soluble and has good physical property as it becomes gel rapidly and turns liquefied instantaneously upon shaking which is very desirable to be used pharmaceutically as thickening agent, gelling agent and film-forming agent in addition to its desirable adhesive property. PG gel as well as PG film preparations were found to possess good wound healing activity on pig and dog skin (Chansiripornchai et al. (2005), Acta Horticulturae 679, 37-43; Chansiripornchai et al. (2006), Acta Pharmacol Sin Suppl. 1,1-489,324). PG preparation was shown in Table 1 to kill Streptococci, the pathogens causing bovine mastitis, to a great extent of over 90% within 1 minute upon exposure.

Table 2 showed that 1% w/v PG preparation induced highest phagocytic activity of bovine mammary leukocytes (BMLS) compared to control. In Table 3, PG at concentration of 0.5 and 1% w/v induced accumulation of total leukocytes in treated mice higher than that of control and values were much lower (p≦0.05) than that of 3% thioglycollate which causes inflammation used as positive control. The result indicates that PG appears to have immuno-stimulating activity on cells of innate immune system in bovine mammary gland as well as in mice at an appropriate concentration of 0.5-1% w/v. Table 4 shows that PG teat dip preparation effectively protected dairy cows against contagious bacteria causing mastitis. Table 5 shows that PG teat dip used in the field effectively protect dairy cows from mastitis infection. This invention describes antibacterial preparations comprises mainly polysaccharide gel from Durian fruit-rind prepared as antiseptic product to be used as post-milking teat dip and teat seal for dairy cows.

Durian fruits are harvested and fruit-rind is dried and used. Polysaccharide gel is extracted from dried fruit-rind of durian by boiling in water for a predetermined period of time. One kilogram of chopped fresh fruit-hull is dried in hot air oven at 70° C. for 8-10 hr, a total of two hundred grams of dried fruit-hulls is obtained. Dried residue is suspended in 25-30 volume by weight of hot distilled water and heated to mild boiling for 40 minutes and filtered. The process is repeated 1-2 times, filtrates are pooled. Water extract of polysaccharide gel is concentrated by rotary evaporating at 70° C. under reduced pressure and precipitated using acidified aqueous ethanol. Precipitate is filtered, dried and pulverized according to Pongsamart and Panmaung (1998) to obtain dry pale beige-color powder designated herein as “PG”. PG is used to prepare antibacterial preparations, i.e. post-milking teat dip preparations for dairy cows.

### EXAMPLE 1

Post-milking Teat Dip Preparations

| | |
|---|---|
| PG | 2-3% w/v |
| Humectant | 5-20% w/v |
| Co-solvent | 5-20% w/v |
| Co-bactericidal agent(s) | 0-10% w/v |
| Physiological Solution | q.s. ad |

### EXAMPLE 2

PG Teat Dip Preparation

| | |
|---|---|
| PG | 2.5% w/v |
| Glycerin | 10% w/v |
| Propylene Glycol | 10% w/v |
| Ringer's Solution | q.s. ad |

### EXAMPLE 3

PG Teat Antiseptic Preparations

| | |
|---|---|
| PG | 2.5% w/v |
| Glycerin | 5-10% w/v |
| Propylene Glycol | 5-10% w/v |
| Povidone iodine or iodophors | 0.5-1% w/v |
| Physiological Solution | q.s. ad |

### EXAMPLE 4

Teat Seal Preparations

| | |
|---|---|
| PG | 2-3% w/v |
| Co-bactericidal agent(s) | 0-5% w/v |
| Thickening agent(s) | 3-10% w/v |
| Distilled water or Physiological Solution | q.s. ad |

### EXAMPLE 5

PG Teat Seal Preparations

| | |
|---|---|
| PG | 2.5% w/v |
| CMC or MC or HPMC | 5-10% w/v |
| Distilled water | q.s. ad |

### EXAMPLE 6

Antiseptic Teat Seal Gel

| | |
|---|---|
| PG | 2.5% w/v |
| Povidone Iodine or iodophors | 1-3% w/v |
| CMC or MC or HPMC | 5-10% w/v |
| Distilled water | q.s. ad |

PG Teat Dip preparation was tested against various field isolates of bacteria causing mastitis in dairy cows and found to kill most of those pathogenic bacteria as the number of colony forming unit of all tested pathogenic bacteria decreased to near zero count within 2-4 hours.

PG Teat Dip preparation was used to dip teat of dairy cows in experimental challenge test with contagious bacteria causing mastitis. It was found to effectively prevent mastitis infection. In addition, it showed good protection against mastitis infection as well as good healing of teat-end when used in the field

It will be understood that modifications can be made in the above description without departing from the scope of this invention by one of ordinary skill in the art. It is accordingly intended that all matter contained in the above description be interpreted as descriptive and illustrative rather than in a limiting sense. It is also to be understood that the following claims are intended to cover all of the generic and specific features of the invention as described herein, and all statements of the scope of the invention which, as a matter of language, might be said to fall therebetween.

TABLE 1
Reduction rate of bovine mastitis Streptococci isolates upon
exposure to teat dip prepared from PG.

| | Reduction rate (%) | | | | |
|---|---|---|---|---|---|
| Time | S. | S. | | | S. |
| (min) | agalactiae | dysagalactiae | S. uberis | S. bovis | acidominimus |
| 0 | 0 | 0 | 0 | 0 | 0 |
| 1 | 92.00 | 92.35 | 90.67 | 96.67 | 93.75 |
| 30 | 98.76 | 96.64 | 99.71 | 99.74 | 98.88 |
| 60 | 99.88 | 99.76 | 99.85 | 99.89 | 99.72 |
| 120 | 99.95 | 99.87 | 99.97 | 99.94 | 99.94 |
| 240 | 99.99 | 99.97 | 100.00 | 100.00 | 99.98 |
| 480 | 100.00 | 99.99 | 100.00 | 100.00 | 99.99 |

TABLE 2
Effect of PG isolated from durian rinds on phagocytic activity of
BMLs by NBT reduction assay. (n = 10).

| | Percentage of phagocytic activity |
|---|---|
| Groups | (mean ± S.E.) |
| Control | −4.83^{b,c} ± 1.41 |
| 0.5% PG | 4.97^{b} ± 3.87 |
| 1% PG | 38.66^{a} ± 4.65 |
| 2.5% PG | −10.87^{c} ± 2.95 |

^{a, b and c}= significantly different (p < 0.05).

TABLE 3
Effect of PG on peritoneal leukocyte count (×10⁶ cell/ml) in mice after
intraperitoneal injection.

| | Leukocyte total | Number of | Number of |
|---|---|---|---|
| | counts | Neutrophil | Lymphocyte |
| Groups | (mean ± S.E.) | (mean ± S.E.) | (mean ± S.E.) |
| NSS | 1.90^{d} ± 0.15 | 0.88^{d} ± 0.13 | 0.97 ± 0.14 |
| 0.5% PG | 13.00^{b} ± 1.40 | 12.12^{b} ± 1.49 | 0.88 ± 0.25 |
| 1% PG | 9.17^{b,c} ± 1.13 | 7.95^{b,c} ± 1.18 | 1.18 ± 0.27 |
| 2.5% PG | 5.53^{c,d} ± 0.84 | 4.06^{c,d} ± 0.99 | 1.74 ± 0.35 |
| 3% thioglycollate | 27.97^{a} ± 2.54 | 27.01^{a} ± 2.59 | 0.86 ± 0.14 |

^{a, b, c and d}= significantly different (p < 0.05).

TABLE 4
Efficacy of PG Teat Dip in protecting against contagious bacteria
causing mastitis in experimental challenge test.

| | | | | Quarter | | |
|---|---|---|---|---|---|---|
| | | Eligible | | day at risk | New IMI per | Reduction |
| Challenge | Experiment | quarters | New | for new | 100 quarter | rate |
| Organisms | Groups | for new IMI | IMI | IMI | days at risk | (%) |
| S. aureus | PG group | 73 | 0 | 505 | 0 | 100* |
| | Control group | 73 | 5 | 492 | 1.02 | |
| | (Undipped) | | | | | |
| S. agalactiae | PG group | 81 | 0 | 640 | 0 | 100* |
| | Control group | 81 | 5 | 626 | 0.8 | |
| | (Undipped) | | | | | |

IMI = Intramammary Infection.
*Significantly different from control (p < 0.05).

TABLE 5
Efficacy of PG Teat Dip in protecting against bacteria causing mastitis in
natural exposure test

| | New IMI per 100 quarter week at risk | | | PG teat dip | | PG teat dip bearing | |
|---|---|---|---|---|---|---|---|
| | (Incidence density) | | | bearing with | | with | |
| | | | Un-dipped | positive control | | negative control | |
| | Iodophor group | | group | group | | group | |
| | (Positive | PG group | (Negative | Rate | | Rate | |
| Type of mastitis pathogens | control) | (Treatment) | control) | difference | (p) | difference | (p) |
| Total bacterial growth samples | 1.95 | 1.47 | 4.17 | 0.48 | (0.449) | 2.70* | (0.037) |
| (%) | | | | | | | |
| Total contagious pathogens (%) | 0.00 | 0.00 | 0.76 | — | — | 0.76 | (0.241) |
| Staphylococcus aureus | 0.00 | 0.00 | 0.38 | — | — | 0.38 | (0.747) |
| Streptococcus agalactiae | 0.00 | 0.00 | 0.00 | — | — | — | — |
| Corynebacterium bovis | 0.00 | 0.00 | 0.38 | — | — | 0.38 | (0.747) |
| Total environmental pathogens | 1.95 | 1.47 | 3.41 | 0.48 | (0.449) | 1.94 | (0.103) |
| (%) | | | | | | | |
| Coagulase-negative | 1.56 | 0.73 | 2.27 | 0.83 | (0.301) | 1.54 | (0.123) |
| staphylococci | | | | | | | |
| Streptococcus spp. | 0.39 | 0.73 | 0.38 | −0.34 | (0.530) | −0.35 | (0.510) |
| Escherichia coli | 0.00 | 0.00 | 0.00 | — | — | — | — |
| Pseudomonas spp. | 0.00 | 0.00 | 0.00 | — | — | — | — |
| Yeast | 0.00 | 0.00 | 0.76 | — | — | 0.76 | (0.241) |
| No bacterial growth sample | 98.05 | 98.53 | 95.83 | 0.48 | (0.499) | 2.70* | (0.037) |
| (%) | | | | | | | |

IMI = Intramammary Infection.
*Significant difference (p ≦ 0.05)

We claim:

1. A polysaccharide gel having antimicrobial activity and immuno-stimulating to be used as a post-milking teat dip agent where it is extracted from dried fruit-rind of durian comprises steps of mild boiling dried fruit rind in 25-30 volume of water for 40 minutes, filtering, repeating mild boiling residue 1-2 times with 15 volume by weight of water, pooling of filtrates and concentrating by rotary evaporating at 70° C. under reduced pressure and precipitating using acidified aqueous ethanol, collecting precipitate, drying and or pulverizing spray-drying.

2. A post-milking teat dip prepared from polysaccharide gel of claim 1 comprises

| | |
|---|---|
| PG | 2-3% w/v |
| Humectant | 5-20% w/v |
| Co-solvent | 5-20% w/v |
| Co-bactericidal agent(s) | 0-10% w/v |
| Physiological Solution | q.s. ad |

3. PG Teat Dip Preparation prepared from polysaccharide gel of claim 1 comprises

| | |
|---|---|
| PG | 2.5% w/v |
| Glycerin | 10% w/v |
| Propylene Glycol | 10% w/v |
| Ringer's Solution | q.s. ad |

4. PG Teat Antiseptic Preparations prepared from polysaccharide gel of claim 1 comprises

| | |
|---|---|
| PG | 2.5% w/v |
| Glycerin | 5-10% w/v |
| Propylene Glycol | 5-10% w/v |
| Povidone iodine or iodophors | 0.5-1% w/v |
| Physiological Solution | q.s. ad |

5. Teat Seal Preparations prepared from polysaccharide gel of claim 1 comprises

| | |
|---|---|
| PG | 2-3% w/v |
| Co-bactericidal agent(s) | 0-5% w/v |
| Thickening agent(s) | 3-10% w/v |
| Distilled water or Physiological Solution | q.s. ad |

6. PG Teat Seal Preparations prepared from polysaccharide gel of claim 1 comprises

| | |
|---|---|
| PG | 2.5% w/v |
| CMC or MC or HPMC | 5-10% w/v |
| Distilled water | q.s. ad |

7. Antiseptic Teat Seal Gel prepared from polysaccharide gel of claim 1 comprises

| | |
|---|---|
| PG | 2.5% w/v |
| Povidone Iodine or iodophors | 1-3% w/v |
| CMC or MC or HPMC | 5-10% w/v |
| Distilled water | q.s. ad |
